(19) United States
(12) Patent Application Publication
(10) Pub. No.: US 2017/0298381 A1
(43) Pub. Date: Oct. 19, 2017

(54) COMBINATION OF FOUR VIP AND CRY PROTEIN TOXINS FOR MANAGEMENT OF INSECT PESTS IN PLANTS

(71) Applicant: Dow AgroSciences LLC, Indianapolis, IN (US)

(72) Inventors: Kenneth E. Narva, Zionsville, IN (US); Joel J. Sheets, San Luis Obispo, CA (US); Sek Yee Tan, Carmel, IN (US); Vimbai Chikwana, Indianapolis, IN (US)

(73) Assignee: Dow AgroSciences LLC, Indianapolis, IN (US)

(21) Appl. No.: 15/485,353

(22) Filed: Apr. 12, 2017

Related U.S. Application Data
(60) Provisional application No. 62/324,490, filed on Apr. 19, 2016.

(51) Int. Cl. C12N 15/82

(52) U.S. Cl. CPC C12N 15/8286

(57) ABSTRACT

The subject invention includes methods and plants for controlling lepidopteran pests, especially soybean looper (Pseudoplusia includens) and velvet bean caterpillar (Anticarsia gemmatalis) insects. The plants, preferably soybean plants, comprise a combination of Cry1Ca, Cry1Ea, Cry2Aa, and Vip3Ab1 insecticidal proteins. Methods are described for using the plants to delay or prevent the development of resistance by insects.

### CROSS REFERENCE TO RELATED APPLICATION

This application claims the benefit of U.S. Provisional application No. 62/324,490, filed Apr. 19, 2016, entitled “COMBINATION OF FOUR VIP AND CRY PROTEIN TOXINS FOR MANAGEMENT OF INSECT PESTS IN PLANTS”, the disclosure of which is being incorporated by reference.

### TECHNICAL FIELD

The present invention relates generally to the field of molecular biology as applied to agricultural sciences. More particularly, certain embodiments concern methods for the use of DNA segments for insecticidal protein expression in plants. Methods of using nucleic acid segments in the development of plant incorporated protectants in transgenic plant cells and plants are disclosed.

### BACKGROUND OF THE DISCLOSURE

Billions of dollars are spent each year to control insect pests and additional billions are lost to the damage they inflict on commercial row crops. Synthetic organic chemical insecticides have been the primary tools used to control insect pests but biological insecticides, such as the insecticidal proteins derived from Bacillus thuringiensis (B.t.), have played an important role in some areas. The ability to produce insect-resistant plants through transformation with B.t. insecticidal protein genes have revolutionized modern agriculture and heightened the importance and value of insecticidal proteins and their genes.

Several B.t. proteins have been used to create insect-resistant transgenic plants that have been successfully registered and commercialized to date. These include Cry1Ab, Cry1Ac, Cry1F and Cry3Bb in corn, Cry1Ac and Cry2Ab in cotton, and Cry3A in potato. The commercial products expressing these proteins express a single protein except in cases where the combined insecticidal spectrum of two proteins is desired (e.g., Cry1Ab and Cry3Bb in corn combined to provide resistance to lepidopteran pests and rootworm, respectively) or where the independent action of the proteins makes them useful as a tool for delaying the development of resistance in susceptible insect populations (e.g., Cry1Ac and Cry2Ab in cotton combined to provide resistance management for tobacco budworm). See also U.S. Patent Application Publication No. 2009/0313717, which relates to a Cry2 protein plus a Vip3Aa, Cry1F, or Cry1A for control of Helicoverpa zea or Helicoverpa armigera. WO 2009/132850 relates to Cry1F or Cry1A and Vip3Aa for controlling Spodoptera frugiperda. U.S. Patent Application Publication No. 2008/0311096 relates in part to Cry1Ab for controlling Cry1F-resistant ECB. WO 2011/084634A1 relates to Vip3Ab and Cry1Ca for controlling fall armyworm.

The wide-spread use of insect-resistant transgenic plants have give rise to the concern that pest populations will develop resistance to the insecticidal proteins produced by these plants. Several strategies have been suggested for preserving the utility of B.t.-based insect resistance traits which include deploying proteins at a high dose in combination with a refuge, and alternation with, or co-deployment of, different toxins (McGaughey et al. (1998), “B.t. Resistance Management,” Nature Biotechnol. 16:144-146).

The proteins selected for use in an insect resistant management (IRM) stack need to exert their insecticidal effect independently so that resistance developed to one protein toxin does not confer resistance to a second protein toxin (eg., there is no cross resistance to the protein toxins). If, for example, a pest population that is resistant to “Protein A” is sensitive to “Protein B”, one would conclude that there is not cross resistance and that a combination of Protein A and Protein B would be effective in delaying resistance to Protein A alone.

In the absence of resistant insect populations, assessments can be made based on other characteristics related to mechanism of action and cross-resistance potential. The utility of receptor-mediated binding in identifying insecticidal proteins likely to not exhibit cross resistance has been suggested (U.S. Pat. No. 5,866,784). The key predictor of lack of cross resistance inherent in this approach is that the insecticidal proteins do not compete for receptors in a sensitive insect species.

In the event that two B.t-derived. toxins compete for the same receptor, if that receptor mutates in that insect resulting in one of the toxins no longer binding to that receptor and thus is no longer insecticidal against the insect, it might be the case that the insect will also be resistant to the second toxin (which competitively bound to the same receptor). That is, the insect is said to be cross-resistant to both B.t. toxins. However, if two toxins bind to two different receptors, this could be an indication that the insect would not be simultaneously resistant to those two toxins.

B.t. toxins are listed at the website of the official B.t. nomenclature committee (Crickmore et al.; http://www.btnomenclature.info/). There are currently more than 70 main groups of “Cry” toxins (Cry1-Cry74), with additional Cyt toxins and VIP toxins and the like. Many of each numeric group have capital-letter subgroups, and the capital letter subgroups have lower-cased letter sub-subgroups. (Cry1 has A-N, and Cry1A has a-j, for example).

Soybeans grown in Latin America and in the southern portions of North America suffer severe economic damage by a number of different lepidopteran insect pests. Soybean looper (SBL; Pseudoplusia includens) and velvet bean caterpillar (VBC; Anticarsia gemmatalis) are considered principle lepidopteran insect pests of soybean in Brazil. The use of insect resistant traits in transgenic soybeans to combat insect damage is just beginning in Latin America. It is expected that Cry1Ac and Cry1Fa, toxins will be the initial Cry toxins that will be used in soybeans in the early phases of commercialization of this technology. These two Bt toxins are the same Cry toxins used in transgenic maize and cotton for insect resistance. Because of the high usage of these two Bt genes in multiple crops over multiple growing seasons, it is expected that there will exist considerable pressure placed on lepidopteran insects to develop resistance against these toxins. Therefore, developing transgenic crops having new Cry toxins that have different modes of action and different insect gut binding sites beyond those provided by Cry1A and Cry1F classes of toxins would be highly beneficial for use in a soybean insect resistance trait strategy.

### BRIEF SUMMARY OF THE DISCLOSURE

The present invention provides transgenic soybean plants having a combination of four Cry and Vip toxins (Cry1Ca, Cry2Aa, Cry1Ea, and Vip3Ab1) that can be used together to provide broad spectrum insecticidal activity against the primary lepidopteran pests in Latin America. For the first time, we show that these four toxins do not compete for the binding to receptors that bind Cry1Ac or Cry1Fa in either SBL or VBC midgut tissues. These results strongly predict that the aforementioned protein toxins, when expressed in combination as stacked genes in transgenic plants, will provide new modes of action to counteract any existence of Cry1A and Cry1F resistance currently present and will slow down or prevent the development of new Bt resistance against this combination of protein toxins.

The invention includes a transgenic plant comprising DNA encoding Cry1Ca, Cry2Aa, Cry1Ea, and Vip3Ab1 insecticidal protein toxins. The most preferred plant is a soybean plant. The invention further includes a seed of a plant, preferably a soybean plant wherein the soybean seed comprises DNA encoding Cry1Ca, Cry2Aa, Cry1Ea, and Vip3Ab1 insecticidal protein toxins. The invention also covers a plurality of plants in a field comprising non-B.t. refuge plants and a plurality of genetically-modified plants of the invention, wherein said refuge plants comprise between 40% to 5% of all crop plants in said field. The invention further covers a mixture of seeds comprising a plurality of refuge seeds from non-B.t. refuge plants and a plurality of genetically-modified seeds of the invention wherein said refuge seed comprise between 40% to 5% of all the seeds in the mixture. A method of controlling lepidopteran pest comprising contacting the pest with an effective amount of a genetically-modified plant of the invention is also claimed. The invention also includes a method of producing a plant of the invention comprising genetically transforming a plant cell with a genetic expression construct comprising DNA encoding Cry1Ca, Cry2Aa, Cry1Ea, and Vip3Ab1 insecticidal protein toxins.

### BRIEF DESCRIPTION OF THE FIGURES

FIG. 1. Homologous competition of Cry1Ac to displace the binding of ¹²⁵I Cry1Ac from midgut membrane vesicles prepared from soybean looper (Pseudoplusia includens) larvae. The lower curve represents fitting of the data with a single binding site model, and the upper curve is fitting the data with a two binding site model.

FIG. 2. Heterologous competition of four different Cry and Vip toxins, as indicated by the labels in the graph, to displace the binding of ¹²⁵I Cry1Ac from midgut membrane vesicles prepared from soybean looper (P. includens) larvae. The multiphasic curve represents fitting of the homologous displacement results from Cry1Ac for comparison.

FIG. 3. Homologous competition of Cry1Ac to displace the binding of ¹²⁵I Cry1Ac from midgut membrane vesicles prepared from velvet bean caterpillar (A. gemmatalis) larvae. The curve represents fitting of the data with a single binding site model.

FIG. 4. Heterologous competition of four different Cry toxins, as indicated by the labels in the graph, to displace the binding of ¹²⁵I Cry1Ac from midgut membrane vesicles prepared from velvet bean caterpillar (A. gemmatalis) larvae. The black curve represents fitting of the homologous displacement results from Cry1Ac for comparison.

FIG. 5. Homologous competition of Cry1Fa to displace the binding of ¹²⁵I Cry1Fa from midgut membrane vesicles prepared from soybean looper (P. includens) larvae. The black curve represents fitting of the data with a single binding site model.

FIG. 6. Heterologous competition of four different Cry toxins, as indicated by the labels in the graph, to displace the binding of ¹²⁵I Cry1Fa from midgut membrane vesicles prepared from soybean looper (P. includens) larvae. The solid black curve represents fitting of the homologous displacement results from Cry1Fa for comparison.

FIG. 7. Homologous competition of Cry1Fa to displace the binding of ¹²⁵I Cry1Fa from midgut membrane vesicles prepared from velvet bean caterpillar (A. gemmatolis) larvae. The curve represents fitting of the data with a single binding site model.

FIG. 8. Heterologous competition of four different Cry toxins, as indicated by the labels in the graph, to displace the binding of ¹²⁵I Cry1Fa from midgut membrane vesicles prepared from velvet bean caterpillar (A. gemmatalis) larvae. The solid black curve represents fitting of the homologous displacement results from Cry1Fa for comparison.

### DETAILED DESCRIPTION OF THE DISCLOSURE

The subject invention relates in part to the surprising discovery that Cry1Ca, Cry2Aa, Cry1Ea, and Vip3Ab1 do not compete for binding with Cry1Ac or Cry1Fa for binding sites in the gut of soybean looper (Pseudoplusia includens; SBL) or velvet bean caterpillar (Anticarsia gemmatalis; VBC). Thus, a Cry1Ca, Cry2Aa, Cry1Ea, and Vip3Ab1 proteins can be used in resistance management in transgenic soybeans (and other plants; e.g., cotton and, corn for example) to delay or prevent resistance to these proteins alone. The subject combination of proteins can be effective at protecting plants (such as soybean, maize and cotton plants) from damage by Cry-resistant SBL or VBC. That is, one use of the subject invention is to protect soybeans and other economically important plant species from damage and yield loss caused by insect populations that could develop resistance to Cry toxins including, but not limited to, Cry1A or Cry1F.

The subject invention thus teaches an insect resistant management (IRM) stack comprising, but not limited to Cry1Ca, Cry2Aa, Cry1Ea, and Vip3Ab1 to prevent or mitigate the development of resistance by SBL or VBC to any of these proteins.

The present invention provides compositions for controlling lepidopteran pests comprising cells that produce a combination of Cry1Ca, Cry2Aa, Cry1Ea, and Vip3Ab1 insecticidal proteins.

The invention further comprises a host transformed to produce Cry1Ca, Cry2Aa, Cry1Ea, and Vip3Ab1 insecticidal proteins, wherein said host is a microorganism or a plant cell. The subject polynucleotide(s) are preferably in a genetic construct under control of one or more non-Bacillus-thuringiensis promoters. The subject polynucleotide codons can be plant-optimized for enhanced expression in a plant.

It is additionally intended that the invention provides a method of controlling lepidopteran pests comprising contacting said pests or the environment of said pests with an effective amount of a composition that contains a Cry1Ca core toxin-containing protein, Cry2Aa core toxin-containing protein, Cry1Ea core toxin-containing protein, or further contains a Vip3Ab1 toxin-containing protein. Core toxin domains of classical 3 domain B.t. toxins are readily discernible by those of ordinary skill in the art of B.t. crystal toxin insecticidal proteins

An embodiment of the invention comprises soybean or maize plants comprising a plant-expressible gene encoding a Cry1Ca insecticidal protein, a plant-expressible gene encoding a Cry2Aa insecticidal protein, a plant-expressible gene encoding a Cry1Ea insecticidal protein, and a plant-expressible gene encoding a Vip3Ab1 insecticidal protein, and seed of such a plant.

A further embodiment of the invention comprises a maize or soybean plant wherein a plant-expressible gene encoding a Cry1Ca insecticidal protein, a plant-expressible gene encoding a Cry2Aa insecticidal protein, a plant-expressible gene encoding a Cry1Ea insecticidal protein, and a plant-expressible gene encoding a Vip3Ab1 insecticidal protein have been introgressed into said maize or soybean plant, and seed of such a plant.

As described in the Examples, competitive receptor binding studies using radiolabeled Cry1Ac or Cry1Fa protein show that Cry1Ca, Cry1Ea, Cry2Aa and Vip3Ab1 proteins do not compete for binding in SBL and VBC tissues to which Cry1Ac or Cry1Fa binds. These results also indicate that the combination of Cry1Ca, Cry1Ea, Cry2Aa and Vip3Ab1 proteins can be an effective means to mitigate the development of resistance in SBL and VBC populations to these proteins. Thus, based in part on the data described herein, it is thought that co-production (stacking) of the Cry1Ca, Cry1Ea, Cry2Aa and Vip3Ab1 proteins can be used to produce an IRM stack for SBL and VBC.

Other proteins can be added to this combination. For example, the subject invention also relates in part to stacks or “pyramids” of four or more toxins, with Cry1Ca, Cry1Ea, Cry2Aa and Vip3Ab1. In some preferred pyramid embodiments, the selected toxins have multiple separate sites of action against SBL and/or VBC. Some preferred pyramid combinations include the subject proteins plus Cry1F, Cry1D, Cry1B, Cry1E, VIP3Aa, or VIP3B), as the third protein for targeting VBC and SBL. By “separate sites of action,” it is meant any of the given proteins do not cause cross-resistance with each other. These particular stacks would, according to the subject invention, advantageously and surprisingly provide multiple sites of action against VBC and SBL. This can help to reduce or eliminate the requirement for refuge acreage.

We demonstrated that Cry1Ca, Cry1Ea, Cry2Aa and Vip3Ab1 do not compete for the binding sites with Cry1Ac or Cry1Fa in the gut of either SBL or VBC. See also WO/2011/075585 “COMBINED USE OF Vip3Ab AND CRY1Fa FOR MANAGEMENT OF RESISTANT INSECTS.”

Thus, the subject combination of toxins Cry1Ca, Cry1Ea, Cry2Aa and Vip3Ab1 provide non-cross-resistant action against SBL and VBC. The inability of Cry1Ca, Cry1Ea, Cry2Aa, and Vip3Ab1 to compete for the binding of Cry1Ac or Cry1F in the gut of SBL and VBC demonstrates that these six protein toxins (Cry1Ca, Cry1Ea, Cry2Aa, Vip3Ab1, Cry1F, and Cry1Ac) represent Cry toxins that provide 3-4 separate target site interactions within the gut of SBL and VBC. These particular stacks would, according to the subject invention, advantageously and surprisingly provide non-cross-resistant action against SBL and VBC. Furthermore, by the demonstration that these four proteins do not compete with each other, one skilled in the art will recognize that this can help to reduce or eliminate the requirement for refuge acreage. As with the benefit of this disclosure, plants expressing the quadruple combination of Cry1Ca, Cry1Ea, Cry2Aa and Vip3Ab1, will be useful in delaying or preventing the development of resistance in SBL and VBC to the individual or combination of these proteins.

Additional toxins and genes can also be added according to the subject invention. For example, if Cry1Fa or Cry1Ac are stacked with subject proteins (Cry1Fa and Cry1Ac are active against SBL and VBC), adding two additional proteins to this stack wherein the two additional proteins target SBL and/or VBC, would provide at least 3 separate sites of action against these pests. These two added proteins would result in a multi-toxin stack with up to 4 modes of action active against two insects (SBL and VBC).

With Cry1Fa being active against SBL and VBC, Vip3Ab1, Cry2Aa, Cry1Ea, or Cry1Ca plus Cry1Fa would, according to the subject invention, advantageously and surprisingly provide three or more sites of action against SBL and VBC. This can help to reduce or eliminate the requirement for refuge acreage.

Cry1Fa is deployed in the Herculex®, SmartStax™, PowerCore™, and WidesStrike™ products. The subject combination of genes (Vip3Ab1, Cry2Aa, Cry1Ea, and Cry1Ca) could be combined into, for example, a Cry1Fa product such as Herculex®, SmartStax™, and WideStrike™ or Cry1A product such as WideStrike™. Accordingly, the subject 4-toxin combination could be important in reducing the selection pressure on these and other cry protein toxins. The subject 4-toxin combination could thus be used for soybean, corn, and other plants such as cotton; though soybean is preferred. As discussed above, additional cry toxins or RNAi-based insecticides can also be added according to the subject invention.

Cry toxins listed on the website of the official B.t. nomenclature committee (Crickmore et al.; http://www.btnomenclature.info/) and GENBANK can be used to obtain the sequences for any of the genes and proteins disclosed or mentioned herein. Relevant sequences are also available in patents. For example, U.S. Pat. No. 5,188,960 and U.S. Pat. No. 5,827,514 describe Cry1Fa core toxin containing proteins suitable for use in carrying out the present invention. U.S. Pat. No. 6,218,188 describes plant-optimized DNA sequences encoding Cry1Fa core toxin-containing proteins that are suitable for use in the present invention. U.S. Ser. No. 61/284,275 (filed Dec. 16, 2009) provides some truncated Cry1Da proteins that can be used according to the subject invention.

Combinations of proteins described herein can be used to control lepidopteran pests. Adult lepidopteran pests, for example, butterflies and moths, primarily feed on flower nectar and are a significant effector of pollination. Nearly all lepidopteran larvae, i.e., caterpillars, feed on plants, and many are serious pests. Caterpillars feed on or inside foliage or on the roots or stem of a plant, depriving the plant of nutrients and often destroying the plant's physical support structure. Additionally, caterpillars feed on fruit, fabrics, and stored grains and flours, ruining these products for sale or severely diminishing their value. As used herein, reference to lepidopteran pests refers to various life stages of the pest, including larval stages.

Some chimeric toxins of the subject invention comprise a full N-terminal core toxin portion of a B.t. toxin and, at some point past the end of the core toxin portion, the protein has a transition to a heterologous protoxin sequence. The N-terminal, insecticidally active, toxin portion of a B.t. toxin is referred to as the “core” toxin. The transition from the core toxin segment to the heterologous protoxin segment can occur at approximately the toxin/protoxin junction or, in the alternative, a portion of the native protoxin (extending past the core toxin portion) can be retained, with the transition to the heterologous protoxin portion occurring downstream.

As an example, one chimeric toxin of the subject invention, is a full core toxin portion of Cry1Ca, Cry1Ea, or Cry2Aa (roughly the first 600 amino acids) and a heterologous protoxin (the remaining amino acids to the C-terminus). In one preferred embodiment, the portion of a chimeric toxin comprising the protoxin is derived from a Cry1Ab protein toxin.

A person of ordinary skill in this art will appreciate that B.t. toxins, even within a certain class such as Cry1Ea, will vary to some extent in length and the precise location of the transition from core toxin portion to protoxin portion. Typically, the Cry1Ea toxins are about 1150 to about 1200 amino acids in length. The transition from core toxin portion to protoxin portion will typically occur at between about 50% to about 60% of the full length toxin. The chimeric toxin of the subject invention will include the full expanse of this N-terminal core toxin portion. Thus, the chimeric toxin will comprise at least about 50% of the full length of the Cry1B.t. toxin protein. This will typically be at least about 590 amino acids. With regard to the protoxin portion, the full expanse of the Cry1Ab protoxin portion extends from the end of the core toxin portion to the C-terminus of the molecule.

Genes and Toxins.

The genes and toxins useful according to the subject invention include not only the full length sequences disclosed but also fragments of these sequences, variants, mutants, and fusion proteins which retain the characteristic pesticidal activity of the toxins specifically exemplified herein. As used herein, the terms “variants” or “variations” of genes refer to nucleotide sequences which encode the same toxins or which encode equivalent toxins having pesticidal activity. As used herein, the term “equivalent toxins” refers to toxins having the same or essentially the same biological activity against the target pests as the claimed toxins.

As used herein, the boundaries represent approximately 95% (Vip3Ab's Cry1Ea's, Cry1Ca's, and Cry2Aa's), 78% (Vip3A's, Cry1C's, Cry1E's, and Cry2A's), and 45% (Cry1's, Cry2's, and Vip3's) sequence identity, per “Revision of the Nomenclature for the Bacillus thuringiensis Pesticidal Crystal Proteins,” N. Crickmore, D. R. Zeigler, J. Feitelson, E. Schnepf, J. Van Rie, D. Lereclus, J. Baum, and D. H. Dean. Microbiology and Molecular Biology Reviews (1998) Vol 62: 807-813. These cut offs can also be applied to the core toxins only.

It should be apparent to a person skilled in this art that genes encoding active toxins can be identified and obtained through several means. The specific genes or gene portions exemplified herein may be obtained from the isolates deposited at a culture depository. These genes, or portions or variants thereof, may also be constructed synthetically, for example, by use of a gene synthesizer. Variations of genes may be readily constructed using standard techniques for making point mutations. Also, fragments of these genes can be made using commercially available exonucleases or endonucleases according to standard procedures. For example, enzymes such as Bal31 or site-directed mutagenesis can be used to systematically cut off nucleotides from the ends of these genes. Genes that encode active fragments may also be obtained using a variety of restriction enzymes. Proteases may be used to directly obtain active fragments of these protein toxins.

Fragments and equivalents which retain the pesticidal activity of the exemplified toxins are within the scope of the subject invention. Also, because of the redundancy of the genetic code, a variety of different DNA sequences can encode the amino acid sequences disclosed herein. It is well within the skill of a person trained in the art to create these alternative DNA sequences encoding the same, or essentially the same, toxins. These variant DNA sequences are within the scope of the subject invention. As used herein, reference to “essentially the same” sequence refers to sequences which have amino acid substitutions, deletions, additions, or insertions which do not materially affect pesticidal activity. Fragments of genes encoding proteins that retain pesticidal activity are also included in this definition.

A further method for identifying the genes encoding the toxins and gene portions useful according to the subject invention is through the use of oligonucleotide probes. These probes are detectable nucleotide sequences. These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170. Some examples of salt concentrations and temperature combinations are as follows (in order of increasing stringency): 2×SSPE or SSC at room temperature; 1×SSPE or SSC at 42° C.; 0.1×SSPE or SSC at 42° C.; 0.1×SSPE or SSC at 65° C. Detection of the probe provides a means for determining in a known manner whether hybridization has occurred. Such a probe analysis provides a rapid method for identifying toxin-encoding genes of the subject invention. The nucleotide segments which are used as probes according to the invention can be synthesized using a DNA synthesizer and standard procedures. These nucleotide sequences can also be used as PCR primers to amplify genes of the subject invention.

Variant Toxins.

Certain toxins of the subject invention have been specifically exemplified herein. Since these toxins are merely exemplary of the toxins of the subject invention, it should be readily apparent that the subject invention comprises variant or equivalent toxins (and nucleotide sequences coding for equivalent toxins) having the same or similar pesticidal activity of the exemplified toxin. Equivalent toxins will have amino acid homology with an exemplified toxin. This amino acid homology will typically be greater than 75%, preferably be greater than 90%, and most preferably be greater than 95%. The amino acid homology will be highest in critical regions of the toxin which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Below is a listing of examples of amino acids belonging to each class.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the toxin.

Recombinant Hosts.

The genes encoding the toxins of the subject invention can be introduced into a wide variety of microbial or plant hosts. Expression of the toxin gene results, directly or indirectly, in the intracellular production and maintenance of the pesticide. Conjugal transfer and recombinant transfer can be used to create a B.t. strain that expresses both toxins of the subject invention. Other host organisms may also be transformed with one or both of the toxin genes then used to accomplish the synergistic effect. With suitable microbial hosts, e.g., Pseudomonas, the microbes can be applied to the environment of the pest, where they will proliferate and be ingested. The result is control of the pest. Alternatively, the microbe hosting the toxin gene can be treated under conditions that prolong the activity of the toxin and stabilize the cell. The treated cell, which retains the toxic activity, then can be applied to the environment of the target pest.

Where the B.t. toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type microorganisms, provide for stable maintenance and expression of the gene expressing the polypeptide pesticide, and, desirably, provide for improved protection of the pesticide from environmental degradation and inactivation.

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobactenum, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobactenium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of methods is available for introducing a B.t. gene encoding a toxin into a microorganism host under conditions which allow for stable maintenance and expression of the gene. These methods are well known to those skilled in the art and are described, for example, in U.S. Pat. No. 5,135,867, which is incorporated herein by reference.

Treatment of Cells.

Bacillus thuringiensis or recombinant cells expressing the B.t. toxins can be treated to prolong the toxin activity and stabilize the cell. The pesticide microcapsule that is formed comprises the B.t. toxin or toxins within a cellular structure that has been stabilized and will protect the toxin when the microcapsule is applied to the environment of the target pest. Suitable host cells may include either prokaryotes or eukaryotes, normally being limited to those cells which do not produce substances toxic to higher organisms, such as mammals. However, organisms which produce substances toxic to higher organisms could be used, where the toxic substances are unstable or the level of application sufficiently low as to avoid any possibility of toxicity to a mammalian host. As hosts, of particular interest will be the prokaryotes and the lower eukaryotes, such as fungi.

The cell will usually be intact and be substantially in the proliferative form when treated, rather than in a spore form, although in some instances spores may be employed.

Treatment of the microbial cell, e.g., a microbe containing the B.t. toxin gene or genes, can be by chemical or physical means, or by a combination of chemical and/or physical means, so long as the technique does not deleteriously affect the properties of the toxin, nor diminish the cellular capability of protecting the toxin. Examples of chemical reagents are halogenating agents, particularly halogens of atomic no. 17-80. More particularly, iodine can be used under mild conditions and for sufficient time to achieve the desired results. Other suitable techniques include treatment with aldehydes, such as glutaraldehyde; anti-infectives, such as zephiran chloride and cetylpyridinium chloride; alcohols, such as isopropyl and ethanol; various histologic fixatives, such as Lugol iodine, Bouin's fixative, various acids and Helly's fixative (See: Humason, Gretchen L., Animal Tissue Techniques, W. H. Freeman and Company, 1967); or a combination of physical (heat) and chemical agents that preserve and prolong the activity of the toxin produced in the cell when the cell is administered to the host environment. Examples of physical means are short wavelength radiation such as gamma-radiation and X-radiation, freezing, UV irradiation, lyophilization, and the like. Methods for treatment of microbial cells are disclosed in U.S. Pat. Nos. 4,695,455 and 4,695,462, which are incorporated herein by reference.

The cells generally will have enhanced structural stability which will enhance resistance to environmental conditions. Where the pesticide is in a proform, the method of cell treatment should be selected so as not to inhibit processing of the proform to the mature form of the pesticide by the target pest pathogen. For example, formaldehyde will crosslink proteins and could inhibit processing of the proform of a polypeptide pesticide. The method of treatment should retain at least a substantial portion of the bio-availability or bioactivity of the toxin.

Characteristics of particular interest in selecting a host cell for purposes of production include ease of introducing the B.t. gene or genes into the host, availability of expression systems, efficiency of expression, stability of the pesticide in the host, and the presence of auxiliary genetic capabilities. Characteristics of interest for use as a pesticide microcapsule include protective qualities for the pesticide, such as thick cell walls, pigmentation, and intracellular packaging or formation of inclusion bodies; survival in aqueous environments; lack of mammalian toxicity; attractiveness to pests for ingestion; ease of killing and fixing without damage to the toxin; and the like. Other considerations include ease of formulation and handling, economics, storage stability, and the like.

Growth of Cells.

The cellular host containing the B.t. insecticidal gene or genes may be grown in any convenient nutrient medium, where the DNA construct provides a selective advantage, providing for a selective medium so that substantially all or all of the cells retain the B.t. gene. These cells may then be harvested in accordance with conventional ways. Alternatively, the cells can be treated prior to harvesting.

The B.t. cells producing the toxins of the invention can be cultured using standard art media and fermentation techniques. Upon completion of the fermentation cycle the bacteria can be harvested by first separating the B.t. spores and crystals from the fermentation broth by means well known in the art. The recovered B.t. spores and crystals can be formulated into a wettable powder, liquid concentrate, granules or other formulations by the addition of surfactants, dispersants, inert carriers, and other components to facilitate handling and application for particular target pests. These formulations and application procedures are all well known in the art.

Formulations.

Formulated bait granules containing an attractant and spores, crystals, and toxins of the B.t. isolates, or recombinant microbes comprising the genes obtainable from the B.t. isolates disclosed herein, can be applied to the soil. Formulated product can also be applied as a seed-coating or root treatment or total plant treatment at later stages of the crop cycle. Plant and soil treatments of B.t. cells may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least 1% by weight and may be 100% by weight. The dry formulations will have from about 1-95% by weight of the pesticide while the liquid formulations will generally be from about 1-60% by weight of the solids in the liquid phase. The formulations will generally have from about 10² to about 10⁴ cells/mg. These formulations will be administered at about 50 mg (liquid or dry) to 1 kg or more per hectare.

The formulations can be applied to the environment of the lepidopteran pest, e.g., foliage or soil, by spraying, dusting, sprinkling, or the like.

Plant Transformation.

A preferred recombinant host for production of the insecticidal proteins of the subject invention is a transformed plant. Non-totipotent plants cells are also an object of the present invention and may be transformed with the subject genes to achieve similar results. Genes encoding B.t. toxin proteins, as disclosed herein, can be inserted into plant cells using a variety of techniques which are well known in the art. For example, a large number of cloning vectors comprising a replication system in Escherichia coli and a marker that permits selection of the transformed cells are available for preparation for the insertion of foreign genes into higher plants. The vectors comprise, for example, pBR322, pUC series, M13mp series, pACYC184, inter alia. Accordingly, the DNA fragment having the sequence encoding an individual B.t. toxin protein or the subject toxin combination can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted. The use of T-DNA for the transformation of plant cells has been intensively researched and sufficiently described in EP 120 516, Lee and Gelvin (2008), Hoekema (1985), Fraley et al., (1986), and An et al., (1985), and is well established in the art.

Once the inserted DNA has been integrated in the plant genome, it is relatively stable. The transformation vector normally contains a selectable marker that confers on the transformed plant cells resistance to a biocide or an antibiotic, such as Bialaphos, Kanamycin, G418, Bleomycin, or Hygromycin, inter alia. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the Right and Left T-DNA border regions. They can be transformed directly into Agrobacteria (Holsters et al., 1978). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizo genes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

In a preferred embodiment of the subject invention, plants will be transformed with genes wherein the codon usage has been optimized for plants. See, for example, U.S. Pat. No. 5,380,831, which is hereby incorporated by reference. While some truncated toxins are exemplified herein, it is well-known in the B.t. art that 130 kDa-type (full-length) toxins have an N-terminal half that is the core toxin, and a C-terminal half that is the protoxin “tail.” Thus, appropriate “tails” can be used with truncated/core toxins of the subject invention. See e.g. U.S. Pat. No. 6,218,188 and U.S. Pat. No. 6,673,990. In addition, methods for creating synthetic B.t. genes for use in plants are known in the art (Stewart and Burgin, 2007). One non-limiting example of a preferred transformed plant is a fertile maize plant comprising a plant expressible gene encoding a Vip3Ab1 protein, further comprising a second plant expressible gene encoding a Cry1Ca protein, further comprising a third plant expressible gene encoding a Cry1Ea protein, and still further comprising a fourth plant expressible gene encoding a Cry2Aa protein.

Transfer, or introgression, of the Cry1Ca-, Cry1Ea-, Cry2Aa- and Vip3Ab1-determined trait(s) into elite soy lines can be achieved by sexual out-crossing using conventional breeding methods. Introgression, of the subject toxin combination into inbred maize lines can be achieved by recurrent selection breeding, for example by backcrossing. In this case, a desired recurrent parent is first crossed to a donor inbred (the non-recurrent parent) that carries the appropriate gene(s) for the Cry1Ca-, Cry1Ea-, Cry2Aa- and Vip3Ab1-determined traits. The progeny of this cross is then mated back to the recurrent parent followed by selection in the resultant progeny for the desired trait(s) to be transferred from the non-recurrent parent. After three, preferably four, more preferably five or more generations of backcrosses with the recurrent parent with selection for the desired trait(s), the progeny will be heterozygous for loci controlling the trait(s) being transferred, but will be like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376). Insect Resistance Management (IRM) Strategies. Roush, for example, outlines two-toxin strategies, also called “pyramiding” or “stacking,” for management of insecticidal transgenic crops. (The Royal Society. Phil. Trans. R. Soc. Lond. B. (1998) 353, 17771786).

On their website, the United States Environmental Protection Agency (epa.gov/oppbppd1/biopesticides/pips/bt_corn_refuge_2006.htm) publishes the following requirements for providing non-transgenic (e.g., non-B.t. refuge plants) refuges, which is a section of non-B.t. plants) for use with transgenic crops producing a single B.t. protein active against pests.
“The specific structured requirements for corn borer-protected Bt (Cry1Ab or Cry1F) corn products are as follows: Structured refuges: 20% non-Lepidopteran Bt corn refuge in Corn Belt; 50% non-Lepidopteran Bt refuge in Cotton Belt. Blocks Internal (i.e., within the Bt field), External (i.e., separate fields within ½ mile (¼ mile if possible) of the Bt field to maximize random mating). In-field Strips, Strips must be at least 4 rows wide (preferably 6 rows) to reduce the effects of larval movement”

In addition, the National Corn Growers Association, on their website: (ncga.com/insect-resistance-management-fact-sheet-bt-corn) also provides similar guidance regarding the refuge requirements. For example: “Requirements of the Corn Borer IRM:
Plant at least 20% of your corn acres to refuge hybrids
In cotton producing regions, refuge must be 50%
Must be planted within ½ mile of the refuge hybrids
Refuge can be planted as strips within the Bt field; the refuge strips must be at least 4 rows wide
Refuge may be treated with conventional pesticides only if economic thresholds are reached for target insect
Bt-based sprayable insecticides cannot be used on the refuge corn
Appropriate refuge must be planted on every farm with Bt corn”

As stated by Roush et al. (on pages 1780 and 1784 right column, for example), stacking or pyramiding of two different proteins each effective against the target pests and with little or no cross-resistance can allow for use of a smaller refuge. Roush suggests that for a successful stack, a refuge size of less than 10% refuge, can provide comparable resistance management to about 50% refuge for a single (non-pyramided) trait. For currently available pyramided B.t. corn products, the U.S. Environmental Protection Agency requires significantly less (generally 5%) structured refuge of non-B.t. corn be planted than for single trait products (generally 20%).

There are various ways of providing the IRM effects of a refuge, including various geometric planting patterns in the fields (as mentioned above) and in-bag seed mixtures, as discussed further by Roush et al. (supra), and U.S. Pat. No. 6,551,962.

The above percentages, or similar refuge ratios, can be used for the subject double, triple, or quadruple stacks or pyramids. For triple or quadruple stacks with three or four sites of action against a single target pest, a goal would be zero refuge (or less than 5% refuge, for example). This is particularly true for commercial acreage—of over 10 acres for example.

All patents, patent applications, provisional applications, and publications referred to or cited herein are incorporated by reference in their entirety to the extent they are not inconsistent with the explicit teachings of this specification.

Unless specifically indicated or implied, the terms “a”, “an”, and “the” signify “at least one” as used herein.

Following are examples that illustrate procedures for practicing the invention. These examples should not be construed as limiting. All percentages are by weight and all solvent mixture proportions are by volume unless otherwise noted. All temperatures are in degrees Celsius.

### Examples

### Example 1—Production and Trypsin Processing of Cry1Ca, Cry1Ea, Cry2Aa and Vip3Ab1 Proteins

The genes encoding the Cry1Ca, Cry1Ea, and Cry2Aa pro toxins were expressed in Pseudomonas fluorescens expression strains and the full length proteins isolated as insoluble inclusion bodies. The washed inclusion bodies were solubilized by stirring at 37° C. in buffer containing 20 mM CAPS buffer, pH 11, +10 mM DDT, +0.1% 2-mercaptoethanol, for 2 hrs. The solution was centrifuged at 27,000×g for 10 min. at 37° C. and the supernatant treated with 0.5% (w/v) TCPK treated trypsin (Sigma). This solution was incubated with mixing for an additional 1 hr. at room temperature, filtered, then loaded onto a Pharmacia Mono Q™ 1010 column equilibrated with 20 mM CAPS pH 10.5. After washing the loaded column with 2 column volumes of buffer, the truncated toxin was eluted using a linear gradient of 0 to 0.5 M NaCl in 20 mM CAPS in 15 column volumes at a flow rate of 1.0 ml/min. Purified trypsin truncated Cry proteins eluted at about 0.2-0.3 M NaCl. The purity of the proteins was checked by SDS PAGE and with visualization using Coomassie brilliant blue dye. In some cases, the combined fractions of the purified toxin were concentrated and loaded onto a Superose 6 column (1.6 cm dia., 60 cm long), and further purified by size exclusion chromatography. Fractions comprising a single peak of the monomeric molecular weight were combined, and concentrated, resulting in a preparation more than 95% homogeneous for a protein having a molecular weight of about 60,000 kDa.

Processing of Vip3Ab1 was achieved in a similar manner starting with the purified full length 85 kDa protein. The protein (12 mg) was dialyzed into 50 mM sodium phosphate buffer, pH 8.4, then processed by adding 1 mg of solid trypsin and incubating for 1 hr at room temperature. The solution was loaded onto a MonoQ™ anion exchange column (1 cm dia., 10 cm. long), and eluted with a linear gradient of NaCl from 0 to 500 mM in 20 mM sodium phosphate buffer, pH 8.4 over 7 column volumes. Elution of the protein was monitored by SDS-PAGE. The major processed band had a molecular weight of 65 kDa, as determined by SDS-PAGE using molecular weight standards for comparison.

### Example 2—Insecticidal Activity of Cry1Ca, Cry1Ea, Cry2Aa, and Vip3Ab1 on SBL and VBC

B.t. insecticidal toxins Cry1Ca, Cry1Ea, Cry2Aa, and Vip3Ab1 were demonstrated to be active on Lepidopteran species including Pseudoplusia includens (SBL) and Anticarsia gemmatalis (VBC).

Sample Preparation and Bioassays.

Inclusion body preparations in 10 mM CAPS pH10 were diluted appropriately in 10 mM CAPS, pH 10, and all bioassays contained a control treatment consisting of this buffer, which served as a background check for mortality.

Protein concentrations in bioassay buffer were estimated by gel electrophoresis using BSA to create a standard curve for gel densitometry, which was measured using a BioRad imaging system (Fluor-S MultiImager™ with Quantity One software version 4.5.2). Proteins in the gel matrix were stained with Coomassie Blue stain and destained before reading.

Purified proteins were tested for insecticidal activity in bioassays conducted with neonate lepidopteran larvae on artificial insect diet. Larvae of SBL and VBC were hatched from eggs obtained from a colony maintained by a commercial insectary (Benzon Research Inc., Carlisle, Pa.).

These bioassays were conducted in 128-well plastic trays specifically designed for insect bioassays (C-D International, Pitman, N.J.). Each well contained 1.0 mL of multi-species Lepidoptera diet (Southland Products, Lake Village, Ark.). A 40 μL aliquot of protein sample was delivered by pipette onto the 1.5 cm² diet surface of each well (26.7 μL/cm²). Cry protein concentrations were calculated as the amount (ng) of protein per square centimeter (cm²) of surface area in the well. The treated trays were held in a fume hood until the liquid on the diet surface had evaporated or was absorbed into the diet.

Within a few hours of eclosion, individual larvae were picked up with a moistened camel hair brush and deposited on the treated diet, one larva per well. The infested wells were then sealed with adhesive sheets of clear plastic, vented to allow gas exchange (C-D International, Pitman, N.J.). Bioassay trays were held under controlled environmental conditions (28° C., ˜60% Relative Humidity, 16:8 [Light:Dark]) for 5 days, after which the total number of insects exposed to each protein sample and the number of dead insects were recorded. Percent mortality was calculated for each treatment.

TABLE 2
Results of bioassay tests of insecticidal proteins
on SBL and VBC, measuring mortality

| Protein | SBL Mortality | VBC Mortality |
|---|---|---|
| Cry1Ac | +++ | +++ |
| Cry1Fa | +++ | +++ |
| Cry1Ca | +++ | +++ |
| Cry1Ea | +++ | +++ |
| Cry2Aa | +++ | ++ |
| Vip3Ab1 | ++ | + |

For Mortality +++ = LC₅₀ <100 ng/cm²; ++ = LC₅₀ between 100-1,000 ng/cm² and + = LC₅₀ between 1,000 and 3,000 ng/cm².

### Example 3—Iodination of Cry1Ca, Cry1Ea, Cry2Aa Core Toxin Protein

The radiolabeled Cry1Ca, Cry1Ea, Cry2Aa proteins were characterized by SDS-PAGE and visualized by phosphor-imaging to validate that the radioactivity measured was covalently associated with the Cry1Ca, Cry1Ea, Cry2Aa core toxin proteins. Coomassie stained SDS-PAGE gels were imaged by wrapping them in Mylar™ film (12 μm thick), and exposing them under a Molecular Dynamics (Sunnyvale, Calif.) storage phosphor screen (35 cm×43 cm) for 1 hour. The plates were developed using a Molecular Dynamics Storm 820 phosphor-imager and the image analyzed using ImageQuant™ software. Some radioactivity was detectable in the gel region well below the Cry1Ca, Cry1Ea, Cry2Aa core toxin proteins band. These radioactive contaminants likely represent small peptides probably associated in the truncated Cry1Ca, Cry1Ea, Cry2Aa proteins due to the action of the trypsin used to cleave the protein to its core structure.

### Example 4—Competitive Binding Assays to BBMVs from SBL and VBC with Core Toxin Proteins of Cry1Ca, Cry1Ea, Cry2Aa, and Vip3Ab1

Homologous and heterologous competition binding assays were conducted using 150 μg/mL BBMV protein and 2 nM of the 125I-radiolabeled Cry1Ac or Cry1F core toxin protein. Concentrations of the homologous competitive non-radiolabeled Cry1Ac or Cry1F core toxin protein added to the reaction mixture were 0.1, 1, 10, 100, and 1000 nM. The heterologous trypsin truncated Cry1Ca, Cry1Ea, or Cry2Aa or full length Vip3Ab1 protein was tested at 0.1, 1, 10, 100, and 1000 nM and the proteins were added at the same time as the radioactive Cry1Ac or Cry1F core toxin protein to assure true binding competition. Incubations were carried out for 1 hr at 28° C. and the amount of 125I-labeled Cry1Ac or Cry1F core toxin protein unbound to the BBMV's (that is, not bound to an insect receptor protein) is separated from bound protein by centrifugation of the BBMV mixture at 16,000×g for 8 min, and removing the supernatant from the resulting pellet. The pellet is washed three times with ice cold binding buffer (PBS; 11.9 mM Na₂HPO₄, 137 mM NaCl, 2.7 mM KCl, pH7.4 plus 0.1% bovine serum albumin; Sigma-Aldrich, St. Louis, Mo.) to completely remove any remaining unbound ¹²⁵I labeled Cry1Ac or Cry1F. The bottom of the centrifuge tube was cut out and the protein pellet contained within this section placed in a 13×100 mm glass culture tube and counted in a gamma counter for 10 minutes to obtain the amount of bound radioactivity contained in the pellet fraction. The amount of radioactivity in the bound protein fraction provides an indication of the amount of Cry protein bound to the insect receptor (total binding). Non-specific binding was represented by the counts obtained in the pellet in the presence of 1,000 nM of non-radiolabeled Cry1Ac or Cry1F core toxin protein. The amount of radiolabeled Cry1Ac or Cry1F specifically bound to the BBMV (specific binding) was measured by subtracting the level of total binding from non-specific binding. One hundred percent total binding was considered to be the amount of binding in the absence of any competitor Cry1Ac or Cry1F core toxin protein. The data is expressed as percent of specific bound ¹²⁵I Cry1Ac or Cry1F versus concentration of competitive unlabeled ligand.

### Example 5—Summary of Results

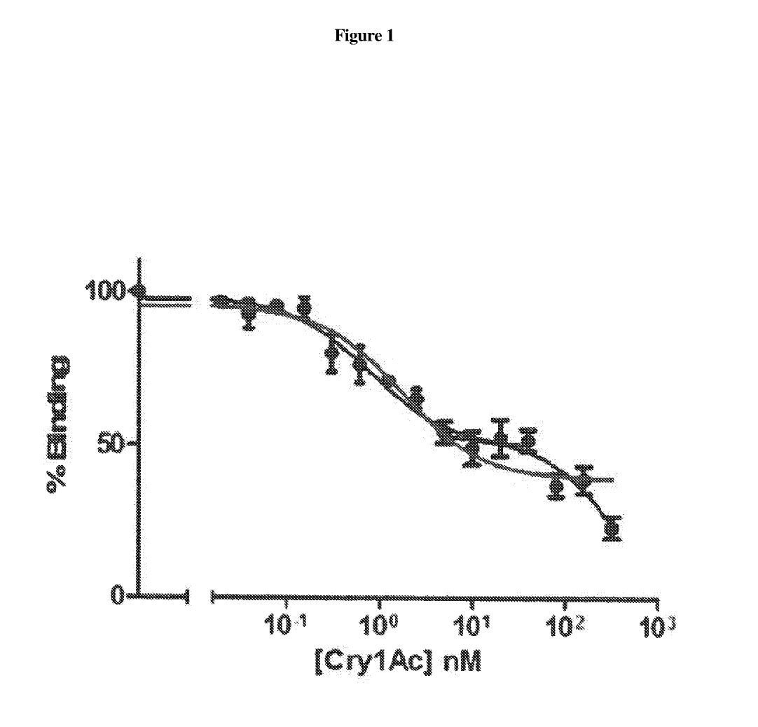

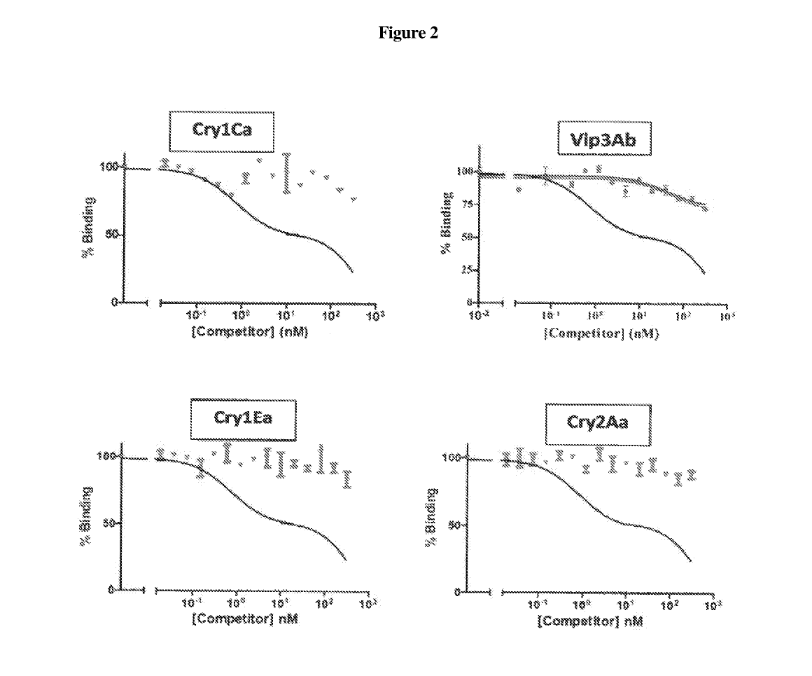

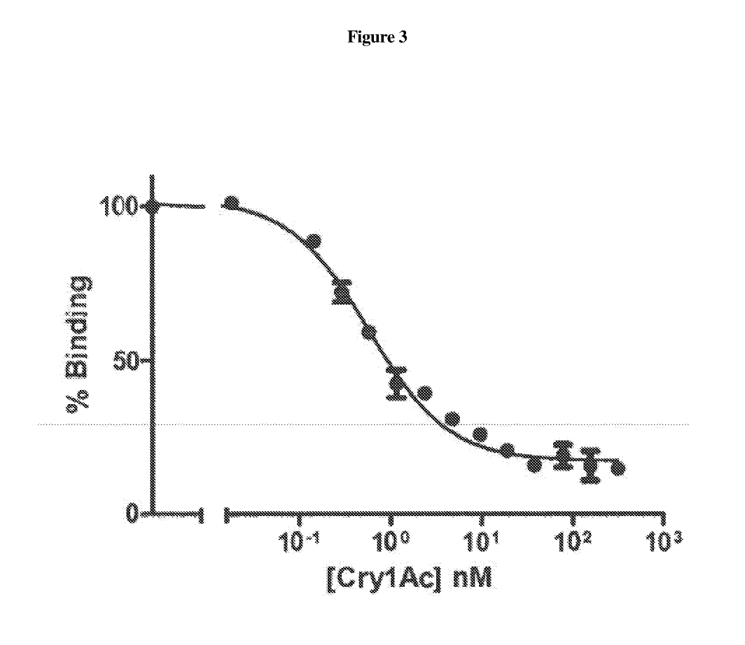

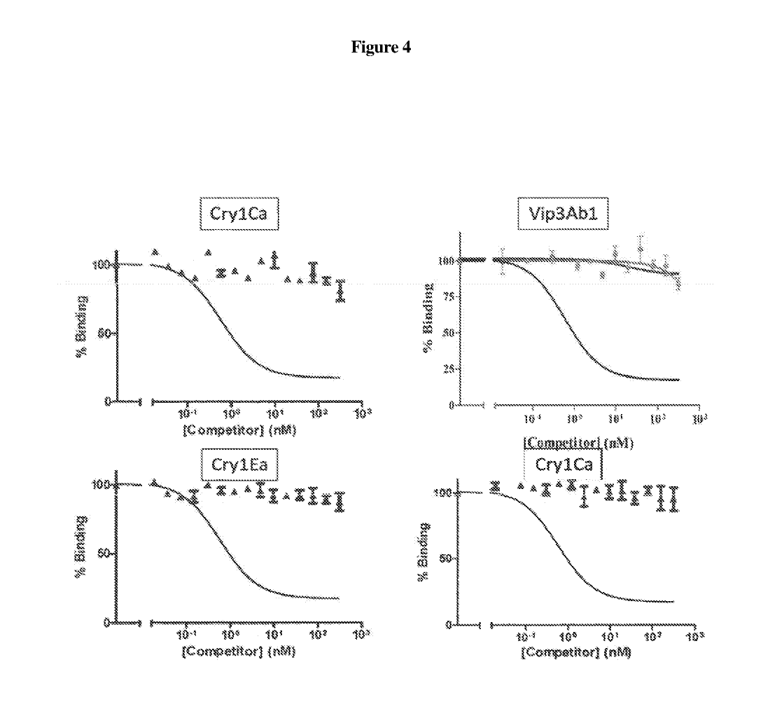

The results (FIGS. 1 and 3) show that the homologous unlabeled Cry1Ac protein effectively displaced the radiolabeled Cry1Ac core toxin protein from specifically binding to the BBMV proteins in a dose dependent manner. Vip3Ab1 did not displace bound ¹²⁵I-labeled Cry1Ac core toxin protein from its receptor protein(s) at either of the concentrations shown (0.1, 1, 10, 100, or 1,000 nM). The highest concentration of Vip3Ab1 tested (1,000 nM) represents 500-fold greater concentration than the radiolabeled Cry1Ac used in the assay, demonstrating that Vip3Ab1 does not effectively compete with the binding of radiolabeled Cry1Ac in SBL or VBC BBMV. Cry1Ca did not displace bound ¹²⁵I-labeled Cry1Ac core toxin protein from its receptor protein(s) at either of the concentrations shown (0.1, 1, 10, 100, or 1,000 nM). The highest concentration of Cry1Ca tested (1,000 nM) represents 500-fold greater concentration than the radiolabeled Cry1Ac used in the assay, demonstrating that Cry1Ca does not effectively compete with the binding of radiolabeled Cry1Ac in SBL or VBC BBMV. Cry1Ea did not displace bound ¹²⁵I-labeled Cry1Ac core toxin protein from its receptor protein(s) at either of the concentrations shown (0.1, 1, 10, 100, or 1,000 nM). The highest concentration of Cry1Ea tested (1,000 nM) represents 500-fold greater concentration than the radiolabeled Cry1Ea used in the assay, demonstrating that Cry1Ea does not effectively compete with the binding of radiolabeled Cry1Ac in SBL or VBC BBMV. Cry2Aa did not displace bound ¹²⁵I-labeled Cry1Ac core toxin protein from its receptor protein(s) at either of the concentrations shown (0.1, 1, 10, 100, or 1,000 nM). The highest concentration of Cry2Aa tested (1,000 nM) represents 500-fold greater concentration than the radiolabeled Cry2Aa used in the assay, demonstrating that Cry2Aa does not effectively compete with the binding of radiolabeled Cry1Ac in SBL or VBC BBMV (FIGS. 2 and 4).

FIG. 1 is a dose response curve for the displacement of ¹²⁵I radiolabeled fluorescein-5-maleimide trypsin-truncated Cry1Ac in BBMV's from Pseudoplusia includens (SBL) larvae. The figure shows the ability of non-labeled Cry1Ac () to displace the labeled Cry1Ac in a dose dependent manner in the range from 0.1 to 1,000 nM. The chart plots the percent of specifically bound labeled Cry1Ac (total bound minus non-specific bound) versus the concentration of the non-radiolabeled ligands added. The inability of non radiolabeled Vip3Ab1 (▴) at 0.1, 1, 10, 100 and 1,000 nM to displace the specifically bound radiolabeled Cry1Ac is shown (FIG. 2). The inability of non radiolabeled Cry1Ca (▴) at 0.1, 1, 10, 100 and 1,000 nM to displace the specifically bound radiolabeled Cry1Ac is shown (FIG. 2). The inability of non radiolabeled Cry1Ea (▴) at 0.1, 1, 10, 100 and 1,000 nM to displace the specifically bound radiolabeled Cry1Ac is shown (FIG. 2). The inability of unlabeled Cry2Aa (▴) at 0.1, 1, 10, 100 and 1,000 nM to displace the specifically bound radiolabeled Cry1Ac is shown (FIG. 2).

FIG. 3 is a dose response curve for the displacement of ¹²⁵I radiolabeled fluorescein-5-maleimide trypsin-truncated Cry1Ac in BBMV's from Anticarsia gemmatalis (VBC) larvae. The figure shows the ability of non-labeled Cry1Ac () to displace the labeled Cry1Ac in a dose dependent manner in the range from 0.1 to 1,000 nM. The chart plots the percent of specifically bound labeled Cry1Ac (total bound minus non-specific bound) versus the concentration of the non-radiolabeled ligands added. The inability of non radiolabeled Vip3Ab1 (▴) at 0.1, 1, 10, 100 and 1,000 nM to displace the specifically bound radiolabeled Cry1Ac is shown (FIG. 4). The inability of non radiolabeled Cry1Ca (▴) at 0.1, 1, 10, 100 and 1,000 nM to displace the specifically bound radiolabeled Cry1Ac is shown (FIG. 4). The inability of non radiolabeled Cry1Ea (▴) at 0.1, 1, 10, 100 and 1,000 nM to displace the specifically bound radiolabeled Cry1Ac is shown (FIG. 4). The inability of unlabeled Cry2Aa (▴) at 0.1, 1, 10, 100 and 1,000 nM to displace the specifically bound radiolabeled Cry1Ac is shown (FIG. 4).

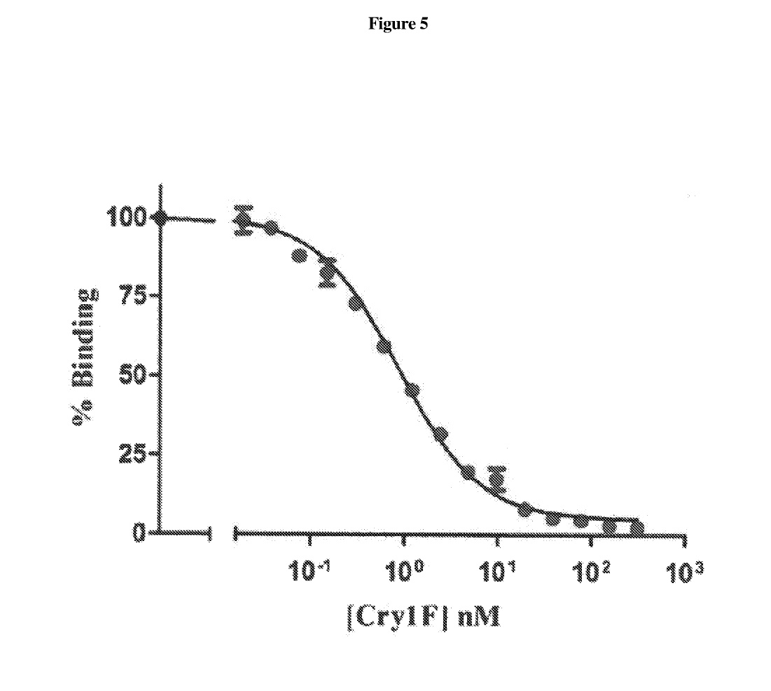

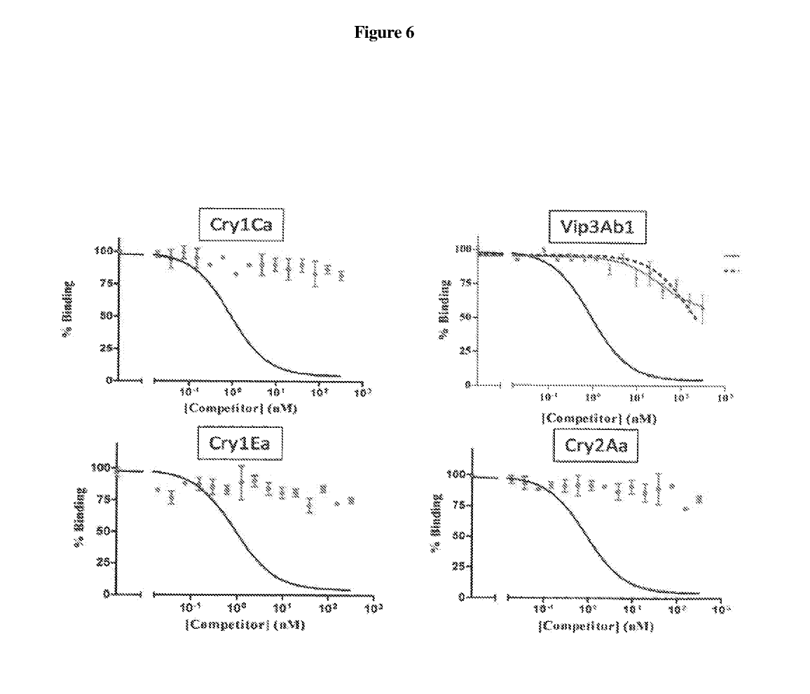

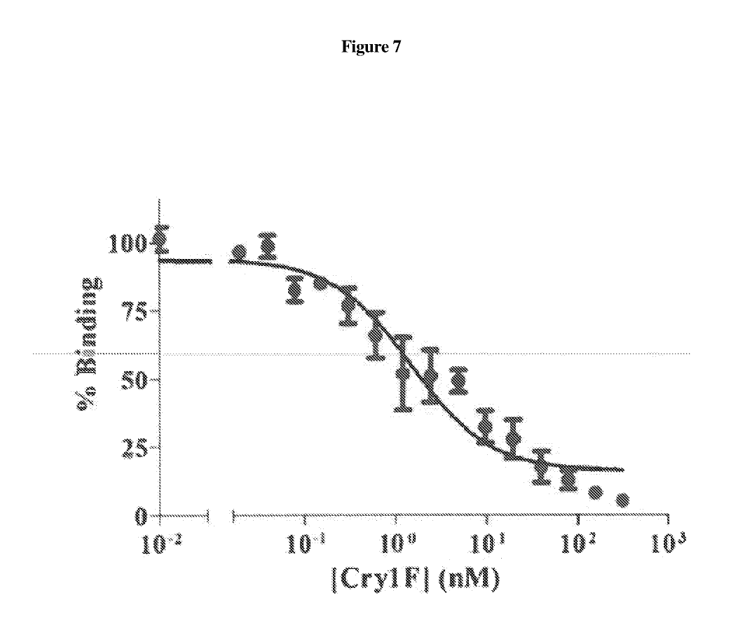

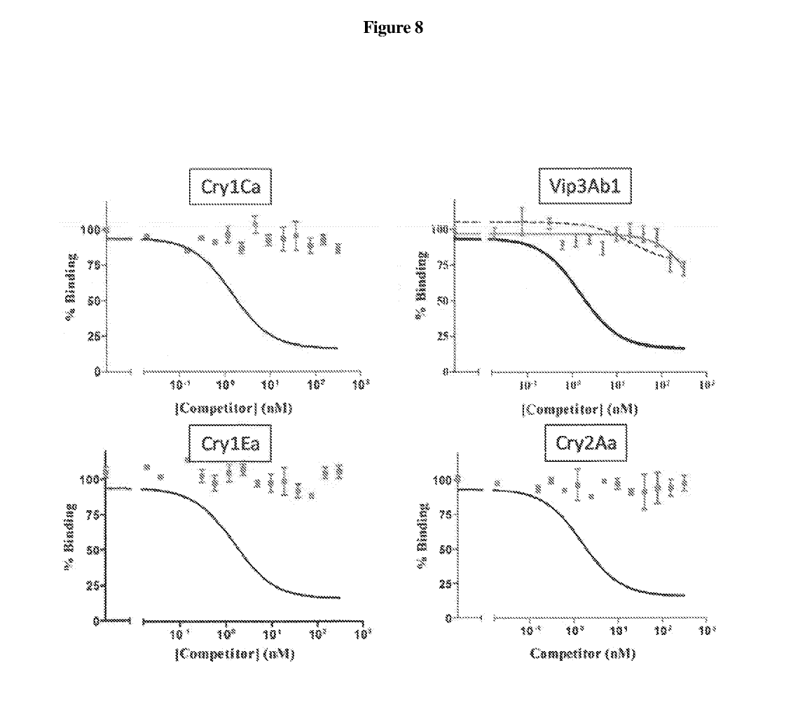

The results (FIGS. 5 and 7) show that the homologous unlabeled Cry1F protein effectively displaced the radiolabeled Cry1F core toxin protein from specifically binding to the BBMV proteins in a dose dependent manner. Vip3Ab1 did not displace bound ¹²⁵I-labeled Cry1F core toxin protein from its receptor protein(s) at either of the concentrations shown (0.1, 1, 10, 100, or 1,000 nM). The highest concentration of Vip3Ab1 tested (1,000 nM) represents 500-fold greater concentration than the radiolabeled Cry1F used in the assay, demonstrating that Vip3Ab1 does not effectively compete with the binding of radiolabeled Cry1F in SBL or VBC BBMV. Cry1Ca did not displace bound ¹²⁵I-labeled Cry1F core toxin protein from its receptor protein(s) at either of the concentrations shown (0.1, 1, 10, 100, or 1,000 nM). The highest concentration of Cry1Ca tested (1,000 nM) represents 500-fold greater concentration than the radiolabeled Cry1F used in the assay, demonstrating that Cry1Ca does not effectively compete with the binding of radiolabeled Cry1F in SBL or VBC BBMV. Cry1Ea did not displace bound ¹²⁵I-labeled Cry1F core toxin protein from its receptor protein(s) at either of the concentrations shown (0.1, 1, 10, 100, or 1,000 nM). The highest concentration of Cry1Ea tested (1,000 nM) represents 500-fold greater concentration than the radiolabeled Cry1Ea used in the assay, demonstrating that Cry1Ea does not effectively compete with the binding of radiolabeled Cry1F in SBL or VBC BBMV. Cry2Aa did not displace bound ¹²⁵I-labeled Cry1F core toxin protein from its receptor protein(s) at either of the concentrations shown (0.1, 1, 10, 100, or 1,000 nM). The highest concentration of Cry2Aa tested (1,000 nM) represents 500-fold greater concentration than the radiolabeled Cry2Aa used in the assay, demonstrating that Cry2Aa does not effectively compete with the binding of radiolabeled Cry1F in SBL or VBC BBMV (FIGS. 6 and 8).

FIG. 5 is a dose response curve for the displacement of ¹²⁵I radiolabeled fluorescein-5-maleimide trypsin-truncated Cry1F in BBMV's from Pseudoplusia includens (SBL) larvae. The figure shows the ability of non-labled Cry1F () to displace the labeled Cry1F in a dose dependent manner in the range from 0.1 to 1,000 nM. The chart plots the percent of specifically bound labeled Cry1F (total bound minus non-specific bound) versus the concentration of the non-radiolabeled ligands added. The inability of non radiolabeled Vip3Ab1 (▴) at 0.1, 1, 10, 100 and 1,000 nM to displace the specifically bound radiolabeled Cry1F is shown (FIG. 6). The inability of non radiolabeled Cry1Ca (▴) at 0.1, 1, 10, 100 and 1,000 nM to displace the specifically bound radiolabeled Cry1F is shown (FIG. 6). The inability of non radiolabeled Cry1Ea (▴) at 0.1, 1, 10, 100 and 1,000 nM to displace the specifically bound radiolabeled Cry1F is shown (FIG. 6). The inability of non radiolabeled Cry2Aa (▴) at 0.1, 1, 10, 100 and 1,000 nM to displace the specifically bound radiolabeled Cry1F is shown (FIG. 6).

FIG. 7 is a dose response curve for the displacement of ¹²⁵I radiolabeled fluorescein-5-maleimide trypsin-truncated Cry1F in BBMV's from Anticarsia gemmatalis (VBC) larvae. The figure shows the ability of non-labled Cry1F () to displace the labeled Cry1F in a dose dependent manner in the range from 0.1 to 1,000 nM. The chart plots the percent of specifically bound labeled Cry1F (total bound minus non-specific bound) versus the concentration of the non-radiolabeled ligands added. The inability of non radiolabeled Vip3Ab1 (▴) at 0.1, 1, 10, 100 and 1,000 nM to displace the specifically bound radiolabeled Cry1F is shown (FIG. 8). The inability of non radiolabeled Cry1Ca (▴) at 0.1, 1, 10, 100 and 1,000 nM to displace the specifically bound radiolabeled Cry1F is shown (FIG. 8). The inability of non radiolabeled Cry1Ea (▴) at 0.1, 1, 10, 100 and 1,000 nM to displace the specifically bound radiolabeled Cry1F is shown (FIG. 8). The inability of non radiolabeled Cry2Aa (▴) at 0.1, 1, 10, 100 and 1,000 nM to displace the specifically bound radiolabeled Cry1F is shown (FIG. 8).

### REFERENCES

An et al., (1985). “New cloning vehicles for transformation of higher plants.” EMBO J 4(2): 277-284.
Crickmore, N., Zeigler, D. R., Feitelson, J., Schnepf, E., Van Rie, J., Lereclus, D., Baum, J., and Dean, D. H. (1998), “Revision of the Nomenclature for the Bacillus thuringiensis Pesticidal Crystal Proteins,” Microbiology and Molecular Biology Reviews 62: 807-813.
Fraley, R. T., Rogers, S. G., Horsch, R. B., Gelvin, S. B. (1986), “Genetic transformation in higher plants,” Critical review in Plant Sci. 4 (1): 1-46.
Hoekema (1985), “Non-oncogenic plant vectors for use in the agrobacterium binary system.” Plant Mol Biol 5(2): 85-89.
Holsters et al., 1978, “Transfection and transformation of Agrobacterium tumefaciens.” Mol Gen Genet 163(2): 181-187.
Humason, Gretchen L. (1967) Animal Tissue Techniques, W. H. Freeman and Company.
Keller, G. H. and Manak, M. M. (1987) DNA Probes, Stockton Press, New York, N.Y., pp. 169-170.
Lee, L. and Gelvin, S. B. (2008), “T-DNA binary vectors and systems,” Plant physiol. 146 (2): 325-332.
Palmer, M., Buchkremer, M, Valeva, A, and Bhakdi, S. Cysteine-specific radioiodination of proteins with fluorescein maleimide. Analytical Biochemistry 253, 175-179. 1997.
Poehlman, J. M. and Sleper, D. A. (1995) Breeding Field Crops, 4th Ed., 172-175; Fehr (1987) Principles of Cultivar Development, Vol. 1: Theory and Technique, 360-376).
Roush R. T. (1998) Two-toxin strategies for management of insecticidal transgenic crops: can pyramiding succeed where pesticide mixtures have not? (The Royal Society. Phil. Trans. R. Soc. Lond. B. Biol. Sci. 353, 1777-1786).

We claim:

1. A transgenic plant comprising DNA encoding Cry1Ca, Cry2Aa, Cry1Ea, and Vip3Ab1 insecticidal protein toxins.

2. The transgenic plant of claim 1 wherein the plant is chosen from the group consisting of soybean, maize and cotton.

3. The transgenic plant of claim 1 wherein the plant is a soybean plant.

4. Seed of a plant according to claim 1, wherein said seed comprises DNA encoding Cry1Ca, Cry2Aa, Cry1Ea, and Vip3Ab1 insecticidal protein toxins.

5. Seed of a plant according to claim 2, wherein said seed comprises DNA encoding Cry1Ca, Cry2Aa, Cry1Ea, and Vip3Ab1 insecticidal protein toxins.

6. Seed of a plant according to claim 3, wherein said seed comprises DNA encoding Cry1Ca, Cry2Aa, Cry1Ea, and Vip3Ab1 insecticidal protein toxins.

7. A plurality of plants in a field, said plants comprising non-B.t. refuge plants and a plurality of plants according to claim 1, wherein said refuge plants comprise between 40% to 5% of all crop plants in said field.

8. A plurality of plants in a field, said plants comprising non-B.t. refuge plants and a plurality of plants according to claim 2, wherein said refuge plants comprise between 40% to 5% of all crop plants in said field.

9. A plurality of plants in a field, said plants comprising non-B.t. refuge plants and a plurality of plants according to claim 3, wherein said refuge plants comprise between 40% to 5% of all crop plants in said field.

10. The plurality of plants of any one of claims 7 to 9, wherein said refuge plants are arranged in blocks or strips.

11. A mixture of seeds comprising a plurality of refuge seeds from non-B.t. refuge plants and a plurality of seeds of claim 4, wherein said refuge seed comprise between 40% to 5% of all the seeds in the mixture.

12. A mixture of seeds comprising a plurality of refuge seeds from non-B.t. refuge plants and a plurality of seeds of claim 5, wherein said refuge seed comprise between 40% to 5% of all the seeds in the mixture.

13. A mixture of seeds comprising a plurality of refuge seeds from non-B.t. refuge plants and a plurality of seeds of claim 6, wherein said refuge seed comprise between 40% to 5% of all the seeds in the mixture.

14. A method of managing development of resistance to a B.t. toxin by an insect, said method comprising planting seeds to produce a plurality of plants of claim 7 in a field.

15. The method of claim 14 in which the insect is chosen from the group consisting of soybean looper (Pseudoplusia includens) and velvet bean caterpillar (Anticarsia gemmatalis).

16. The method of claim 15 in which the plurality of plants is soybean.

17. A plurality of plants of any one of claims 7 to 9, wherein said plants occupy more than 10 acres.

18. A method of controlling lepidopteran pest, said method comprising contacting said pest with an effective amount of a plant of any one of claims 1 to 3.

19. A method of producing a plant of claim 1, said method comprising genetically transforming a plant cell with a genetic expression construct comprising DNA encoding Cry1Ca, Cry2Aa, Cry1Ea, and Vip3Ab1 insecticidal protein toxins.

20. A method of producing a plant of claim 3, said method comprising genetically transforming a plant cell with a genetic expression construct comprising DNA encoding Cry1Ca, Cry2Aa, Cry1Ea, and Vip3Ab1 insecticidal protein toxins.
